(19) United States
(12) Patent Application Publication
(10) Pub. No.: US 2026/0102311 A1
(43) Pub. Date: Apr. 16, 2026

(54) AMBLYOPIA TRAINING DEVICE

(71) Applicants: Zhuhai ULook MetaBrain Medical Technology Co. Ltd., Zhuhai City (CN); METABRAIN TECHNOLOGY PTE.LTD., SINGAPORE (SG)

(72) Inventors: Wen-Fang LEI, Taoyuan City (TW); Shih-Hsien CHENG, Taoyuan City (TW); Shin-Da LEE, Taichung City (TW)

(21) Appl. No.: 19/194,632

(22) Filed: Apr. 30, 2025

(30) Foreign Application Priority Data
Oct. 10, 2024 (CN) 202411409612.6

(51) Int. Cl. A61H 5/00; A61H 39/00; A61M 21/00

(52) U.S. Cl. CPC A61H 5/005; A61H 39/00; A61M 21/00; A61H 2039/005; A61H 2201/1607; A61M 2021/0027; A61M 2021/005; A61M 2021/0055; A61M 2205/054; A61M 2205/056

(57) ABSTRACT

An amblyopia training device includes a bracket unit, a virtual reality unit including a display headset, a dichoptic training unit with contrast color filters including first and second contrast color filters, a binaural audio stimulation unit including paired speakers for playing binaural beats music, a transcranial stimulation unit connected to the rear side part, and a control unit. The bracket unit includes top and rear side parts, and ears part. The control unit is electrically connected with the display headset, the speaker and the transcranial stimulation unit. The amblyopia training device provided by the invention can stimulate the user in various ways through the display headset, the speaker, the first and second contrast color filters and the transcranial stimulation unit, so as to achieve the synergistical effect of activating the visual center of the user to increase the visual acuity in person with amblyopia.

### BACKGROUND OF THE INVENTION

### Field of the Invention

The invention relates to the technical field of amblyopia therapeutics, in particular to an amblyopia training device.

### Description of the Prior Art

Amblyopia is the sequela of the visual center of visual cortex in brain not being exposed to enough stimulation during its development, so it stops developing. Therefore, how to interact and activate the visual center through multiple non-invasive approaches to increase the visual acuity of amblyopia patients is the focus of the current research.

The present invention is, therefore, arisen to obviate or at least mitigate the above-mentioned disadvantages.

### SUMMARY OF THE INVENTION

The main object of the present invention is to provide an amblyopia training device, which can stimulate the user in a single course of treatment by arranging display headset, a speaker, a first contrast color filter, a second contrast color filter and a transcranial stimulation unit, so as to synergistically activate the visual center of the user and increase the visual acuity of the user.

To achieve the above and other objects, an amblyopia training device is provided, wherein the amblyopia training device includes: a bracket unit corresponding to a user's head shape, and comprising a top side part, a rear side part and two ears part, wherein the two ears part respectively correspond to the user's ears; two ends of the top side part are respectively connected with the two ears part and straddle the user's head upwards; and two ends of the rear side part are respectively connected with the two ears part and straddle the user's occipital bone backwards; a virtual reality unit comprising a display headset connected to the bracket unit and capable of switching between a display mode and a visual photobiomodulatory stimulation mode, wherein when the display headset is in the display mode, the display headset displays a virtual image, and when the display headset is in the visual photobiomodulatory stimulation mode, the display headset displays flashing light with a specific wavelength and a specific frequency; a dichoptic training unit with contrast color filters comprising a frame, a first contrast color filter and a second contrast color filter, wherein the frame is connected to the inner side of the display headset, and the first contrast color filter and the second contrast color filter are respectively connected to the frame to correspond to the two eyes of the user, and the colors of the first contrast color filter and the second contrast color filter are contrastly different; a binaural audio stimulation unit comprising paired speakers, wherein the paired speakers are respectively connected to the two ears part and used for playing binaural beats music, and the binaural beats music has an binaural audio frequency difference; a transcranial stimulation unit connected to the rear side part to correspond to the brain region of the occipital lobe of the user corresponding to the visual center; a control unit electrically connected with the display headset, the speaker and the transcranial stimulation unit, wherein the control unit stores data information of the binaural beats music and the virtual image.

In some embodiments, it also comprises an eye mask unit comprising an eye mask body, two eyes surrounding heating parts and ten eyes surrounding acupoint stimulation parts. The eye mask body is detachably connected to the bracket unit and straddles the eyes of the user forward, the eye mask body is provided with eye holes for exposing the eyes of the user and watching virtual reality with the display headset. The dichoptic training unit with contrast color filters is located between the eye mask body and the display headset, the eyes surrounding heating parts are electrically connected with the control unit, the eyes surrounding heating parts are connected with the eye mask body and used for thermally stimulating the periphery of the user's eyes through a first preset temperature, the eyes surrounding acupoint stimulation parts are electrically connected with the control unit, and eyes surrounding acupoint stimulation parts are connected with the eye mask body and correspond to the user's two Jingming BL1 acupoints, two Cuanzhu BL2 acupoints, two Yuyao EX-HN4, two Sizhukong TE23 acupoints and two Chengqi ST1 acupoints respectively, and the eyes surrounding acupoint stimulation parts are used for heating the user's two Jingming BL1 acupoints, two Cuanzhu BL2 acupoints, two Yuyao EX-HN4 acupoints, two Sizhukong TE23 acupoints and two Chengqi ST1 acupoints respectively at a second preset temperature.

In some embodiments, it also comprises a head laser acupoint stimulation unit, which comprises three head acupoint media, the head laser acupoint stimulation medium are electrically connected with the control unit and used for outputting laser light, and three head laser acupoint stimulation medium are connected with the rear and top side part to correspond to two Fengchi GB20 acupoints and one Baihui GV20 acupoint of the user respectively, that is, the three head laser acupoint stimulation medium are used for physically stimulating the laser light output from the two Fengchi GB20 acupoints and one Baihui GV20 acupoint of the user respectively.

Preferably, the binaural audio frequency difference of the binaural beats music is between 4 Hz and 18 Hz.

Preferably, the virtual image for dichoptic training unit includes a main two contrast color block which two colors matching those of two contrast color filters, a first color block and a second color block, the colors of the main color block and the first color block are different, and the main color block and the first color block partially overlap, the color of that main color block and the second color block are different, the main color block and the second color block partially overlap, the color of the first color block is the same as that of the first color filter, and the color of the second color block is the same as that of the second contrast color filter.

Preferably, the flashing light is with a wavelength of 380 nm-780 nm and a frequency of 5 Hz-45 Hz.

Preferably, the laser wavelength of the head laser acupoint stimulation medium is 650-808 nm.

In some embodiments, it also comprises an exercise bike unit comprising a bike frame, a flywheel and a pedal group, wherein the bike frame is used for the user to ride. The flywheel is rotatably connected to the bike frame, and the pedal group is in transmission connection with the flywheel and is used for being stepped by the user to rotate the flywheel around the central axis of the flywheel.

In some embodiments, the transcranial stimulation unit includes two first magnetic stimulation medium, which are respectively connected to the rear side part to correspond to the P1 position and the P2 position among the positions of the international 10-20 brainwave electrode, and the first magnetic stimulation unit is used for outputting electromagnetic pulses for transcranial magnetic stimulation; The first magnetic stimulation medium comprises a first extension frame, a mounting seat and a first magnetic coil, wherein the first extension frame is connected to the rear side part; the mounting seat has a first end and a second end; the first end is connected to the first extension frame; and the first magnetic coil is connected to the mounting seat and electrically connected with the control unit.

Preferably, the mounting seat comprises a plurality of support layers stacked in sequence along the direction from the first end to the second end, and each support layer is provided with the first magnetic coil; among any two adjacent support layers, the outer contour of one support layer near the first end is larger than that of one support layer far from the first end, and one support layer far from the first end is arranged in the middle of one support layer near the first end.

More preferably, the end face of one end of the support layer far from the first end is a first magnetic surface, and the first magnetic coil encloses a first area around the center of the first magnetic surface on the first magnetic surface; among any two adjacent support layers, one support layer far from the first end is arranged in the first area of one support layer close to the first end.

Preferably, the control unit can control the magnetic coil to emit a plurality of current waves within a predetermined time T, each current wave has a rated current value, the rated current values of the current waves within the predetermined time T are gradually increasing from small to large, and the rated current values are between 1 A and 10 A.

In some embodiments, the transcranial stimulation unit includes two first transcranial electrical stimulation media, and the two first transcranial electrical stimulation media are respectively connected to the rear side part to respectively correspond to the P1 position and P2 position in the international 10-20 EEG electrode position; the first transcranial electrical stimulation media comprises a second extension frame, a first connecting seat and a plurality of first conductive posts, wherein the second extension frame is connected to the rear side part, the first connecting seat is connected to one end of the second extension frame far away from the rear side part, and the first conductive posts are connected to the first connecting seat and electrically connected with the control unit.

In some embodiments, the transcranial stimulation unit comprises a second magnetic stimulation medium, and the second magnetic stimulation medium comprises a shell-shaped shell and a second magnetic coil, wherein the shell-shaped shell is connected to the rear side part, and the magnetic coil is connected to the shell-shaped shell and arranged in a spiral shape.

In some embodiments, the transcranial stimulation unit includes two second transcranial electrical stimulation media, and the two second transcranial electrical stimulation media are respectively connected to the rear side part to respectively correspond to the P1 position and P2 position among the positions of the international 10-20 EEG electrodes; the second transcranial electrical stimulation media comprises a second connecting seat and a second conductive post, wherein the second transcranial electrical stimulation media has a third end and a fourth end, the third end is connected to the bracket unit, and the second conductive post is connected to the fourth end and electrically connected to the control unit; the number of the second conductive posts is multiple, and the second conductive posts are distributed on the end face of the fourth end in parallel; the second conductive column comprises a connecting part and a silicon electrode part, wherein the connecting part is provided with a fifth end and a sixth end, the fifth end is connected to the fourth end, and the silicon electrode part is connected to the sixth end; the number of the silicon electrode part is multiple, and the silicon electrode part are distributed on the end face of the sixth end in parallel.

Compared with the prior, the amblyopia training device provided by the embodiment of the invention has the beneficial effects that:
(1) Through the paired speakers, binaural beats music with binaural audio frequency difference (4-18 Hz) can be played to both ears of the user to activate the visual center in visual cortex of occipital lobe.
(2) The display headset can stimulate the user's eyes with flashing light with specific wavelength and frequency, which is helpful to activate the user's visual center.
(3) By displaying the virtual image for dichoptic training unit and the cooperation of the first color filter and the second contrast color filter with different colors, the amblyopic eye can be trained to be naturally forced to use the amblyopic eye to present an image because of contrast color difference, and the visual center corresponding to the amblyopic eye of the user can be activated.
(4) The brain region of the occipital lobe of the user corresponding to the visual center can be stimulated by the transcranial stimulation unit to help activate the visual center of the user.
(5) The head laser acupoint stimulation unit can increase the blood circulation of the brain and the visual center, so the amblyopia training device provided by the invention can stimulate the user in a single course of treatment through the virtual imagine of display headset for dichoptic training unit with two contrast color filters, the paired speaker with binaural audio stimulation, and the transcranial stimulation unit, so as to synergistically increase the visual acuity of the user via interacting and co-activating the visual center.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a schematic structural diagram of an amblyopia training device provided by the first embodiment of the present invention;

FIG. 2 is a schematic diagram of the control connection structure of an amblyopia training device provided by the first embodiment of the present invention;

FIG. 3 is a partial structural schematic diagram of an amblyopia training device provided by the first embodiment of the present invention;

FIG. 4 is a side view of an amblyopia training device provided by the first embodiment of the present invention;

FIG. 5 is a front view of an amblyopia training device provided by the first embodiment of the present invention;

FIG. 6 is a schematic structural diagram of the first magnetic stimulation medium provided in the first embodiment of the present invention;

FIG. 7 is a schematic diagram of a virtual image displayed by display headset provided in the first embodiment of the present invention;

FIG. 8 illustrates an image displayed when a user watches a virtual image through a first color filter according to the first embodiment of the present invention;

FIG. 9 illustrates an image displayed when a user watches a virtual image through a second contrast color filter according to the first embodiment of the present invention;

FIG. 10 is a waveform diagram of the current applied by the magnetic coil of the control unit provided in the first embodiment of the present invention;

FIG. 11 is a diagram showing the effect relationship between multiple amblyopia training and increasing visual acuity percentage after training by using an amblyopia training device with multiple stimuli provided by the first embodiment of the present invention.

FIG. 12 is a schematic structural diagram of an amblyopia training device provided by the second embodiment of the present invention;

FIG. 13 is a schematic structural diagram of the first transcranial electrical stimulation media provided by the second embodiment of the present invention;

FIG. 14 is a schematic structural diagram of an amblyopia training device provided by the third embodiment of the present invention;

FIG. 15 is a side view of an amblyopia training device provided by the third embodiment of the present invention; and

FIG. 16 is a schematic structural diagram of the second transcranial electrical stimulation media, comb-shaped silicon electrode, provided by the fourth embodiment of the present invention.

### DETAILED DESCRIPTION OF THE PREFERRED EMBODIMENTS

In the following, the specific embodiments of the present invention will be described in further detail with reference to the attached drawings and examples. The following examples are used to illustrate the invention, but are not used to limit the scope of the invention.

In the description of the present invention, it should be understood that the terms “center”, “longitudinal”, “lateral”, “length”, “width” “thickness”, “Up”, “Down”, “Front”, the azimuth or positional relationship indicated by “back”, “left”, “right”, “vertical”, “horizontal”, “top”, “bottom”, “inside” and “outside” is based on the azimuth or positional relationship shown in the attached figures. It is only for the convenience of describing the present invention and simplifying the description, and does not indicate or imply that the referred devices or elements must have a specific orientation, be constructed and operated in a specific orientation, so it cannot be understood as limiting the present invention.

The terms “first” and “second” are only used for descriptive purposes, and cannot be understood as indicating or implying relative importance or implicitly indicating the number of indicated technical features. Therefore, the features defined as “first” and “second” may include one or more of these features explicitly or implicitly.

In the present invention, unless otherwise specified and limited, the terms “installation”, “connection”, “connection” and “fixation” should be broadly understood, for example, they can be fixed connection, detachable connection or integrated. It can be a mechanical connection or an electrical connection, can be directly connected, can also be indirectly connected through an intermediary, can be the internal communication of two elements or the interaction between two elements. For those skilled in the art, the specific meanings of the above terms in the present invention can be understood according to specific situations.

In the present invention, unless otherwise specified and limited, the first feature “above” or “below” the second feature may include direct contact between the first and second features, or it may include that the first and second features are not in direct contact but through another feature contact between them. Moreover, the words “above”, “above” and “above” of the first feature include that the first feature is directly above and obliquely above the second feature, or only indicate that the horizontal height of the first feature is higher than the second feature. The words “under”, “under” and “under” of the first feature include that the first feature is directly below and obliquely below the second feature, or only indicate that the horizontal height of the first feature is smaller than the second feature.

### Embodiment I

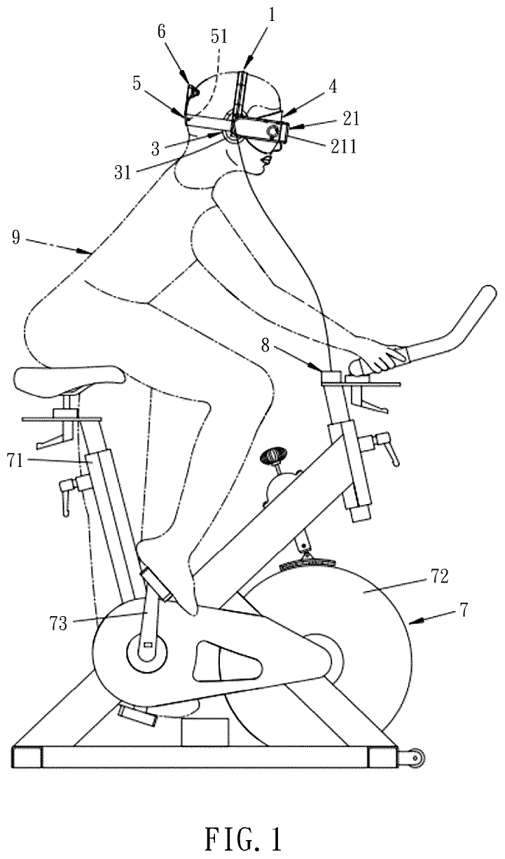

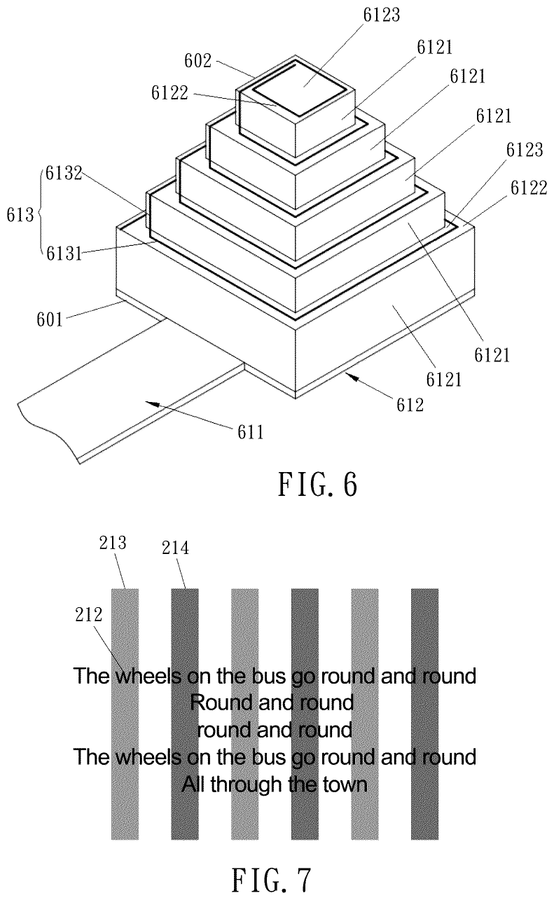

As shown in FIGS. 1-6, an amblyopia training device provided by the first embodiment of the present invention includes a bracket unit 1, a virtual reality unit 2, a dichoptic training unit with contrast color filters 22, a binaural audio stimulation unit 3, an eye mask unit 4, a head laser acupoint stimulation unit 5, a transcranial stimulation unit 6, an exercise bike unit 7 and a control unit 8.

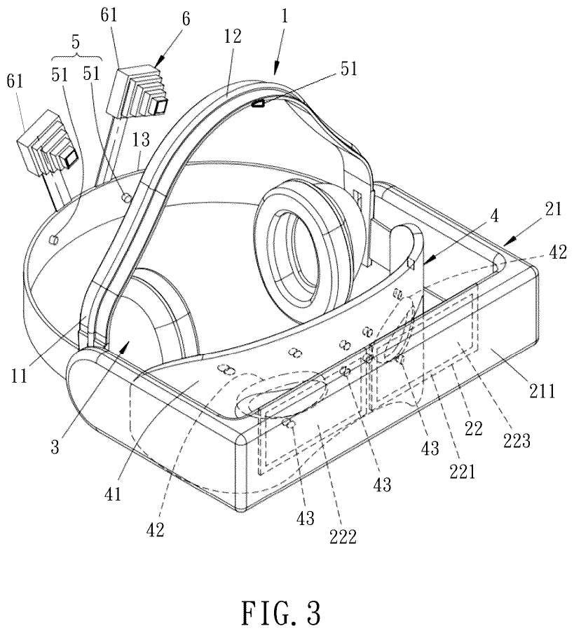

Referring to FIG. 1, FIG. 3-FIG. 5, the bracket unit 1 corresponds to the head shape of a user 9, and includes a top side part 12, a rear side part 13 and two ears part 11, and the two ears part 11 respectively correspond to both ears of the user 9. Two ends of the top side part 12 are respectively connected with the two ears part 11 and straddle the top of the head of the user 9, and two ends of the rear side part 13 are respectively connected with the two ears part 11 and straddle the occipital bone of the user 9.

Referring to FIGS. 1, 3-5, and 7-9, the virtual reality unit 2 includes display headset 21, and the display headset 21 are connected to the bracket unit 1 and can switch between a display mode and a light frequency stimulation mode. When the display headset 21 is in the display mode, the display headset 21 display a virtual image, and when the display headset 21 is in the light frequency stimulation mode, the display headset 21 display flashing light with a specific wavelength and a specific frequency. The user's eyes can be stimulated by flashing light with a specific wavelength and frequency through the visual photobiomodulatory stimulation mode, so as to achieve the synergistical effect of vision training.

The dichoptic training unit with contrast color filters 22 comprises a frame 221, a first contrast color filter 222 and a second contrast color filter 223, wherein the frame 221 is connected to the inside of the display headset 21, The first contrast color filter 222 and the second contrast color filter 223 are respectively connected to the frame 221 to correspond to the eyes of the user 9, and the colors of the first contrast color filter 222 and the second contrast color filter 223 are different.

It can be understood that “the color of the first contrast color filter 222 and the color of the second contrast color filter 223 are different” means that the colors of the first contrast color filter 222 and the second contrast color filter 223 are different.

The virtual image includes a main color object 212, a first color block 213, and a second color block 214. The color of the main color object 212 is different from that of the first color block 213, and the main color object 212 and the first color block 213 partially overlap. The color of the main color object 212 is different from that of the second color block 214, and the main color object 212 and the second color block 214 partially overlap. The color of the first color block 213 is the same as that of the first contrast color filter 222, and the color of the second color block 214 is the same as that of the second contrast color filter 223.

In this embodiment, the main color object 212 is a piece of black characters, and partially overlaps with the first color block 213 and the second color block 214. The colors of the first contrast color filter 222 and the first color block 213 are red, and the colors of the second contrast color filter 223 and the second color block 214 are green.

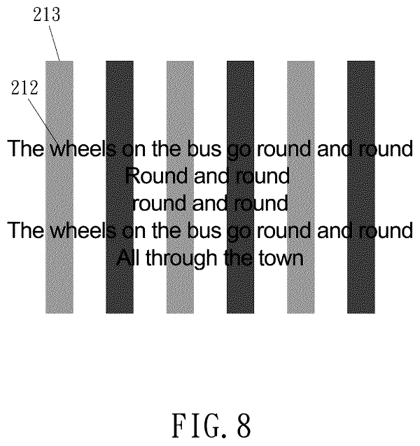

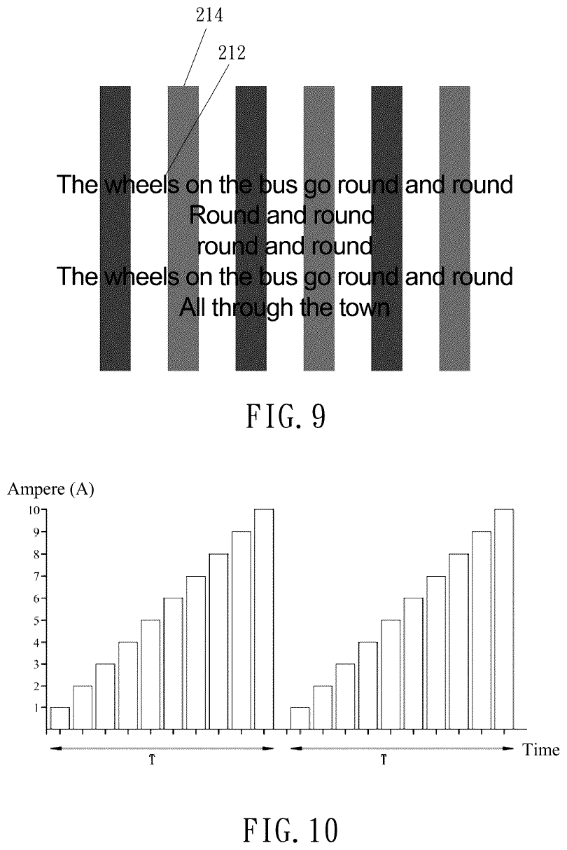

By adopting a virtual image with a main color object 212, a first color block 213 and a second color block 214 with different colors, when one eye of the user 9 is viewed through the first contrast color filter 222, The second color block 214 and the main color object 212 in the overlapped part are black and cannot be recognized, but only the first color block 213 and other parts of the main color object 212 can be seen. The virtual image displayed by the display headset 21 is as shown in FIG. 7, while the virtual image seen by the user is as shown in FIG. 8. When the other eye of the user 9 looks through the second contrast color filter 223, the first color block 213 and the overlapped main color object 212 are black and cannot be recognized, but only the second color block 214 and other parts of the main color object 212 can be seen. The virtual image displayed by the display headset 21 is as shown in FIG. 7, while the virtual image seen by the user is as shown in FIG. 9. The user 9 will integrate the images seen by both eyes into a complete image of the main color object 212 in his brain by this binocular separation method. Therefore, the visual center of the user's brain can be trained through the cooperation of the virtual image and the dichoptic training unit with contrast color filters 22, and the effect of activating the visual center of the user to increase the visual acuity of amblyopic eye can be achieved.

In this embodiment, the number of the first color blocks 213 is plural, and the number of the second color blocks 214 is plural, and the first color blocks 213 and the second color blocks 214 are distributed alternately.

In this embodiment, the specific wavelength and the specific frequency of flashing light are wavelength between 380 nm and 780 nm and a frequency between 5 Hz and 45 Hz. That is, when the display headset 21 are in the visual photobiomodulatory stimulation mode, the display headset 21 display light with a flashing rate of 5 Hz to 45 Hz and a wavelength of 380 nm to 780 nm.

Referring to FIGS. 1-3, the binaural audio stimulation unit 3 includes paired speakers 31, and the paired speakers 31 are respectively connected to the two ears part 11. Binaural beats music with a specific binaural audio frequency difference can be played through the speaker 31, so as to induce the user's brain waves to induce frequency following response and enter a specific brain wave form, activate the visual center to achieve better visual acuity in amblyopic eye via vision training with binaural beats music.

Preferably, the speaker 31 is used to play the binaural beats music (Binaural beats with Frequency Following Response) to both ears of the user 9. The binaural beats music has a binaural audio frequency difference of 4 Hz-18 Hz, which gradually decreases from the high binaural audio frequency difference of 18 Hz to 4 Hz.

Referring to FIGS. 1-5, the eye mask unit 4 includes an eye mask body 41, eyes surrounding heating parts 42 and eyes surrounding acupoint stimulation parts 43. The eye mask body 41 is detachably connected to the bracket unit 1 and straddles the eyes of the user 9 forward. The eye mask body 41 is provided with eye holes 411 for exposing the eyes of the user 9, and the eyes surrounding heating parts 42 are electrically connected with the control unit 8. The eyes surrounding heating parts 42 is connected to the eye mask body 41 and is used for thermally stimulating the eyes of the user 9 through a first preset temperature. The eyes surrounding acupoint stimulation parts 43 are electrically connected with the control unit 8, and the eyes surrounding acupoint stimulation parts 43 are connected with the eye mask body 41 and are used for respectively thermally stimulating the two Jingming BL1 points, two Cuanzhu BL2 acupoints, two Yuyao EX-HN4 acupoints, two Sizhukong TE23 acupoints and two Chengqi ST1 acupoints of the user 9 through a second preset temperature. Through the eye mask unit 4, when the user 9 performs eye training or flashing light of visual photobiomodulatory stimulation through the display headset 21, at the same time, the eyes surrounding heating parts 42 and the eyes surrounding acupoint stimulation parts 43 can respectively perform thermal stimulation on the surrounding of the user's eyes and ten specific eye acupoints, so as to increase the visual nervous activity, relax the eye muscles, and promote the blood circulation around the eyes, thus achieving the better visual acuity via amblyopia training device.

In this embodiment, both ends of the eye mask body 41 are detachably connected to the two ears part 11.

In this embodiment, the first preset temperature is between 40° C. and 45° C.

In this embodiment, the second preset temperature is 50° C.

Preferably, the eyes surrounding heating parts 42 are made of graphene, and the eyes surrounding acupoint stimulation parts 43 are made of graphene.

Referring to FIGS. 1-4, the head laser acupoint stimulation unit 5 includes three head laser acupoint stimulation medium 51, the head laser acupoint stimulation medium 51 is electrically connected to the control unit 8, and the three head laser acupoint stimulation medium 51 are connected to the rear part 13 and top side part 12 and used for physically stimulating the laser output from two Fengchi GB20 acupoints and one Baihui GV20 acupoint of the user 9, respectively. Through the head laser acupoint stimulation medium 51, specific head acupoints can be stimulated by laser, so as to stimulate vision-related function and further improve the visual acuity via amblyopia training device with head laser acupoint stimulation.

In this embodiment, the laser wavelength of the head laser acupoint stimulation medium 51 is 650-808 nm.

Referring to FIGS. 1-6, the transcranial stimulation unit 6 is connected to the rear side part 13 to correspond to the brain region of the occipital lobe of the user 9 corresponding to the visual center, and the transcranial stimulation unit 6 is used for outputting electromagnetic pulses for transcranial magnetic stimulation or outputting current for transcranial electrical stimulation.

In this embodiment, the transcranial stimulation unit 6 includes two first magnetic stimulation medium 61, and the two first magnetic stimulation medium 61 are respectively connected to the rear side part 13 to correspond to the P1 position and the P2 position among the electrode positions of the International 10-20 EEG generator, and the first magnetic stimulation medium 61 are used for outputting electromagnetic pulses.

Preferably, the first magnetic stimulation medium 61 is detachably connected to the rear side part 13, and the first magnetic stimulation medium 61 with different structures can be replaced as needed, and the first magnetic stimulation medium 61 can also be replaced with an electric stimulation medium as needed.

The first magnetic stimulation medium 61 includes a first extension frame 611, a mounting base 612 and a first magnetic coil 613. The first extension frame 611 is connected to the rear side part 13, and the mounting base 612 has a first end 601 and a second end 602 opposite to each other. The first end 601 is connected to the first extension frame 611, and the first magnetic coil 613 is connected to the mounting base 612. The first magnetic coil 613 is electrically connected to the control unit 8 and can be generated when power is applied. Using two magnetic stimulation media to precisely perform transcranial magnetic stimulation on the P1 position and P2 position of the head of the user 9, respectively, is helpful to activate the visual center of the user 9 and increase the visual acuity of the user 9.

The mounting base 612 includes a plurality of support layers 6121 stacked in sequence along the direction from the first end 601 to the second end 602, and each support layer 6121 is provided with a first magnetic coil 613. Among any two adjacent support layers 6121, the outer contour of one support layer 6121 near the first end 601 is larger than that of one support layer 6121 far from the first end 601, and the one support layer 6121 far from the first end 601 is arranged in the middle of one support layer 6121 near the first end 601. In this way, the mounting base 612 can be made into a laminated tower structure that gradually narrows along the first end 601 to the second end 602, so that the support layer 6121 farthest from the first end 601 (that is, the support layer 6121 located on the second end 602) can precisely align with the P1 position or P2 position of the user 9. So that the magnetic force of the first magnetic coil 613 can precisely stimulate the P1 position or P2 position of the user 9 through the second end 602, which further helps to activate the visual center of the user 9 and increase the visual acuity of the user 9.

In this embodiment, the number of support layers 6121 is five. The five support layers 6121 are a first support layer, a second support layer, a third support layer, a fourth support layer and a fifth support layer which are sequentially distributed along the direction from the first end 601 to the second end 602, so that the outer contour of the first support layer is larger than that of the second support layer, and the second support layer is arranged in the middle of the first support layer. The outer contour of the second support layer is larger than that of the third support layer, and the third support layer is arranged in the middle of the second support layer. The outer contour of the third support layer is larger than that of the fourth support layer, and the fourth support layer is arranged in the middle of the third support layer. The outer contour of the fourth support layer is larger than that of the fifth support layer, and the fifth support layer is arranged in the middle of the fourth support layer.

In this embodiment, the support layer 6121 has a square structure, and the length, width and height of the support layer 6121 with the largest outer contour are 3 cm, 3 cm and 1 cm respectively. The support layer 6121 with the smallest outer contour is 1 cm in length, 1 cm in width and 1 cm in height. It can be adapted to the P1 position and P2 position of the head of the user 9, and effectively magnetically stimulate the P1 position and P2 position of the head of the user 9, so as to further help activate the visual center of the user and increase the visual acuity of the user.

The end face of the support layer 6121 far from the first end 601 is a first magnetic surface 6122, and the first magnetic coil 613 surrounds the center of the first magnetic surface 6122 on the first magnetic surface 6122 with a first area 6123. Among any two adjacent support layers 6121, one support layer 6121 far from the first end 601 is arranged in the first area 6123 of one support layer 6121 near the first end 601. In this way, the first magnetic coil 613 can be arranged in a spiral shape around a plurality of support layers 6121 to concentrate electromagnetic pulses, and the first magnetic surface 6122 of one support layer 6121 (i.e., the fifth support layer) located on the second end 602 can be adapted to the P1 position or P2 position of the user. Therefore, the second end 602 can precisely stimulate the concentrated electromagnetic pulse to the P1 position or P2 position of the user 9, which is more conducive to activating the visual center of the user 9 and increasing the visual acuity of the user 9.

Specifically, in any two adjacent support layers 6121, the first magnetic coil 613 is arranged around the outer contour of the support layer 6121 far from the first end 601 on the first magnetic surface 6122 of the support layer 6121 near the first end 601, and extends from the first magnetic surface 6122 of the support layer 6121 near the first end 601 along the outer side wall of the support layer 6121 far away from the first end 601 to the first magnetic surface 6122 of the support layer 6121 far away from the first end 601.

More specifically, the first magnetic coil 613 includes a plurality of surrounding segments 6131 and a plurality of straight line segment 6132. A plurality of surrounding segments 6131 and straight line segment 6132 are alternately connected. The surrounding segments 6131 is arranged on the first magnetic surface 6122 and encloses the first area 6123, and the straight section 6132 is arranged on the outer side wall of the support layer 6121, and the straight section 6132 is the shortest interconnection structure connecting the two first magnetic surfaces 6122. In this way, it can not only adapt to the laminated tower structure of the mounting base 612, but also make the magnetic coil in a spiral-like structure that gradually narrows along the direction from the first end 601 to the second end 602 to concentrate the electromagnetic pulse along the direction from the first end 601 to the second end 602. It is also possible to have the shortest interconnection path between two adjacent surrounding segments 6131 to reduce the energy loss of the magnetic coil so as to more effectively and stably convert electric power into magnetic force, so that the P1 position or P2 position of the user 9 can be more effectively magnetically stimulated, which is more conducive for activating the visual center of the user 9 and better increasing the visual acuity of the user 9.

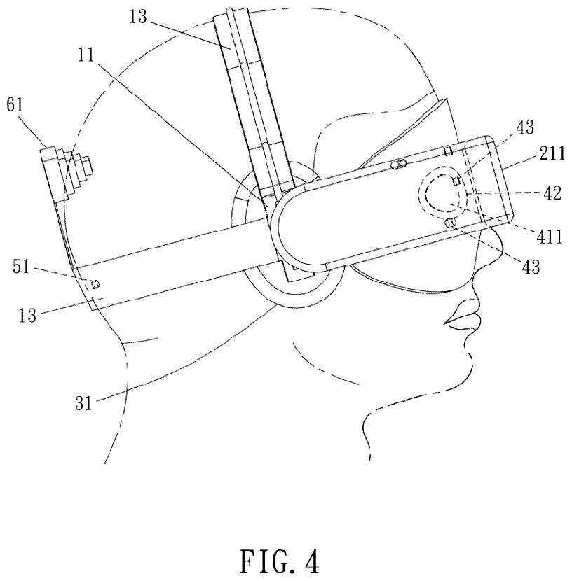

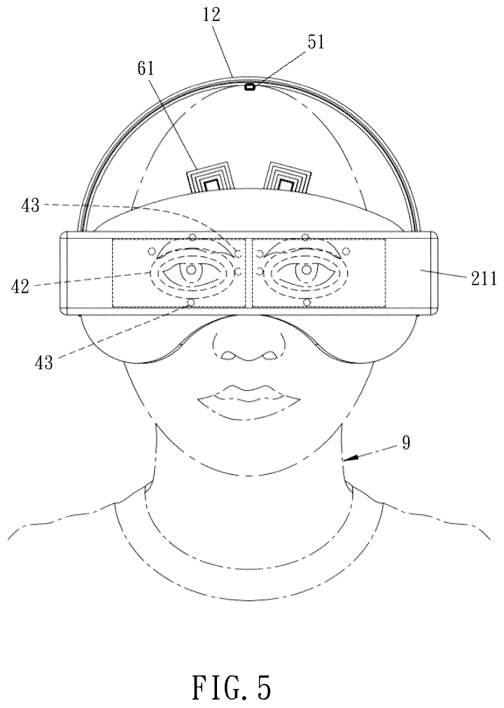

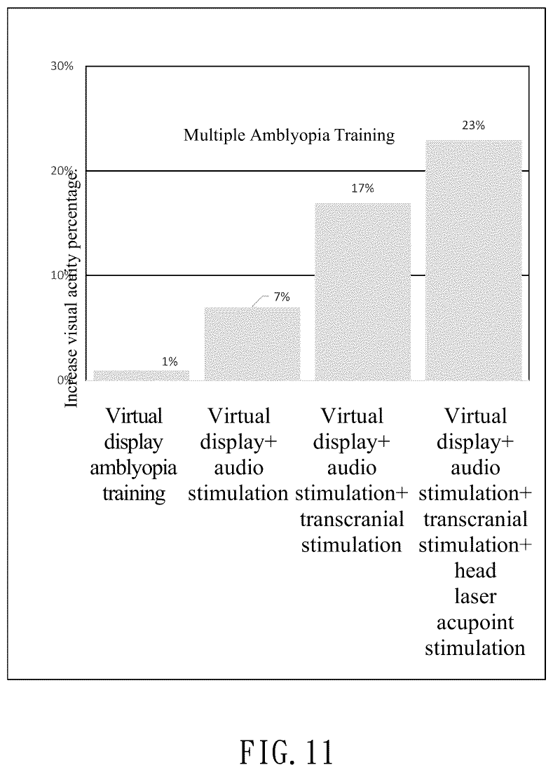

Referring to FIG. 1, FIG. 3, FIG. 4 and FIG. 5, by connecting the virtual reality unit 2, the binaural audio stimulation unit 3, the eye mask unit 4, the head laser acupoint stimulation unit 5 and the transcranial stimulation unit 6 to the bracket unit 1, the bracket unit 1, the virtual reality unit 2, the binaural audio stimulation unit 3, the eye mask unit 4, the head laser acupoint stimulation unit 5 and the transcranial stimulation unit 6 can be combined into wearable training equipment for amblyopians. The wearable training equipment integrates multiple stimuli. Referring to FIG. 11, the amblyopia training device provided by Embodiment 1 is used to train for amblyopians in a single course of treatment. As can be seen from FIG. 11, the more amblyopia training items, the better visual acuity, that is, various units from amblyopia training device can activate the visual center of users to interact and to synergistically increase the visual acuity of amblyopians.

Referring to FIG. 1, the exercise bike unit 7 includes a bike frame 71, a flywheel 72 and a pedal group 73, wherein the bike frame 71 is used for a user to ride, the flywheel 72 is rotatably connected to the bike frame 71, and the pedal group 73 is in transmission connection with the flywheel 72 and is used for a user 9 to step on to rotate the flywheel 72 around the central axis of the flywheel 72.

In this embodiment, the exercise bike unit is electrically connected with the control unit 8. Specifically, the exercise bike unit 7 further includes a power module and an energy conversion module, which are respectively connected with the flywheel and the power module, and the energy conversion module is used for converting the rotational potential energy of the flywheel into electric energy and storing the electric energy in the power module, which is electrically connected with the control unit.

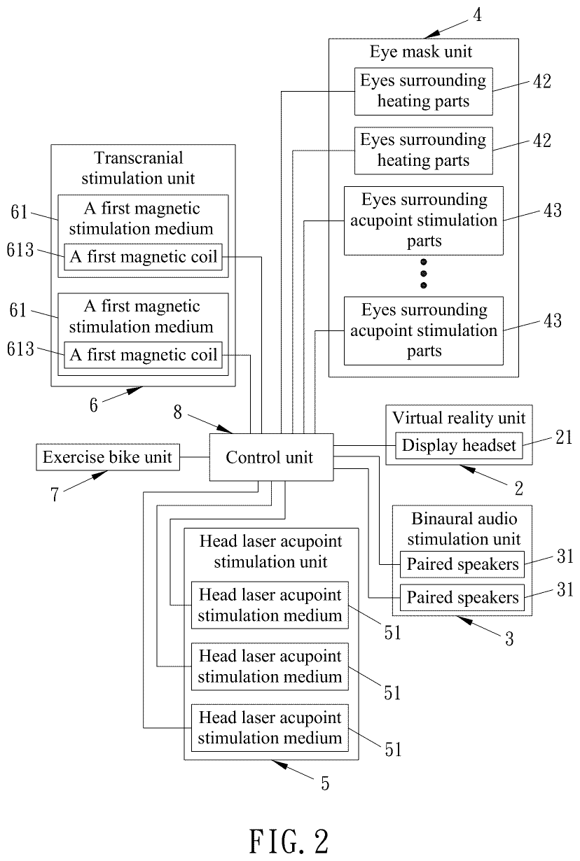

Referring to FIGS. 1 and 2, the control unit 8 is electrically connected with the display headset 21, the speaker 31, the eyes surrounding heating parts 42, the eyes surrounding acupoint stimulation parts 43, the head laser acupoint stimulation medium 51 and the first magnetic stimulation medium 61, and the control unit 8 stores data information of binaural beats music and virtual images. The control unit 8 can also receive power from the power module. The control unit 8 can control the display headset 21 to display a virtual image or display a flashing light with a specific wavelength and frequency. The control unit 8 controls the eyes surrounding heating parts 42 and the eyes surrounding acupoint stimulation parts 43 to generate heat. The control unit 8 controls the head laser acupoint stimulation medium 51 to emit laser light and the first magnetic stimulation medium and the second magnetic stimulation medium to emit magnetic stimulation.

In this embodiment, the control unit 8 is arranged on the exercise bike unit 7, but the control unit 8 can also be arranged on the virtual reality unit 2, and is not limited to this.

Referring to FIG. 10, in this embodiment, the control unit 8 can control the first magnetic coil 613 to emit a plurality of current waves within a predetermined time T, each current wave has a rated current value, and the rated current value of the current waves within the predetermined time T is gradually increasing from small to large, and the rated current value is between 1 A and 10 A.

In some embodiments, the predetermined time T is 1 second, and the number of current waves is 10 groups, but it can also be adjusted to 20 groups every 1 second as required, without limitation.

Referring to FIGS. 1-10, the use method of the amblyopia training device provided by the embodiment of the invention is as follows: Before use, the user 9 first wears the bracket unit 1, then sits on the bike frame 71 and steps on the pedal group 73 to prepare for the training of stepping on the flywheel. During training, the user 9 starts to step on the pedal group 73 for 30 minutes. At this time, the control unit 8 controls the first magnetic stimulation medium 61 to emit magnetic stimulation for 30 minutes to the brain region of the occipital lobe of the user corresponding to the visual center, and simultaneously controls the head acupoint medium 51 to emit laser stimulation for 30 minutes to the corresponding head acupoints, and simultaneously controls the eyes surrounding heating parts 42 and the eyes surrounding acupoint stimulation parts 43 to perform thermal therapy for 30 minutes around the eyes and corresponding eye acupoints. In the first 15 minutes, the control unit 8 controls the display headset 21 to switch to the display mode, and controls the speaker 31 to play binaural beats music. In the next three minutes, the control unit 8 controls the display headset 21 to switch to the visual photobiomodulatory stimulation mode, and controls the speaker 31 to stop playing binaural beats music and play health education content instead. In the last 12 minutes, the control unit 8 controls the display of the display headset 21 to switch to the display mode, and controls the speaker 31 to play the binaural beats music, thus completing the whole set of amblyopia training device.

It should be noted that when the display headset 21 are switched to the visual photobiomodulatory stimulation mode with flashing light, the health education content played by the speaker 31 includes suggestions such as diet, exercise, sleep, bright environmental arrangement, and out-door activity arrangement.

Since the user 9 can activate the visual center by binocular separation when watching the display headset 21, the virtual image is matched with the first contrast color filter 222 and the second contrast color filter 223. It can not only train the vision of amblyopic eyes, but also match the effect of watching with normal eyes at the same time, so as to further integrate binocular vision and help activate the user's visual center to increase visual acuity.

Ten subjects with amblyopia were tested in FIG. 11. After listening to the binaural beats music from virtual reality for 30 minutes, the visual acuity of the tested amblyopia eye increased by 7% on average After listening to binaural beats music from virtual reality, stimulating P1 and P2 brain regions for 30 minutes, the visual acuity of amblyopic eyes increased by around 17% on average. After listening to binaural beats music from virtual reality, stimulating P1 and P2 brain regions, and receiving head laser acupoint stimulation for 30 minutes, the visual acuity of amblyopic eyes increased by 23% on average. Therefore, it can be proved that listening to binaural beats music and stimulating P1 and P2 brain regions at the same time can additively increase visual acuity. Based on animal experiments (tDCS recovers depth perception in adult amblyopic rats and reorganizes visual cortex activity. S Castano, etc. Behav Brain Res. 2019 Sep, 16:370:111941), it is found that magnetic stimulation of P1 and P2 brain regions can increase the remodeling of visual cortical neural activity.

In addition, the influence of exercise and photobiomodulatory stimulation wavelength of 380-780 nm on improving visual acuity can be proved by references of Harrington S, Kearney J, O'Dwyer V. Visual factors associated with physical activity in schoolchildren. Clin Exp Optom. 2022 Aug 11:1-11, so this specification will not be further explained.

To sum up, the amblyopia training device provided by the embodiment of the present invention, through the cooperation of the virtual reality unit 2 and the dichoptic training unit with contrast color filters 22 to activate the visual center, the speaker 31 for playing binaural beats music, the head laser acupoint stimulation unit 5 for providing head laser acupoint stimulation, the transcranial stimulation unit 6 for providing magnetic stimulation, the eye mask unit 4 for providing thermal stimulation and the exercise bike unit 7, can synergistically increase visual acuity in a single course of treatment via multiple stimulation.

### Embodiment II

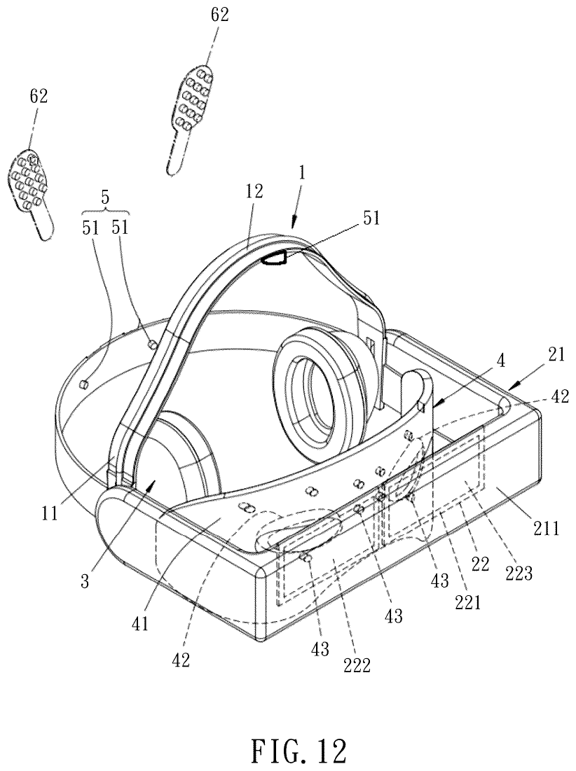

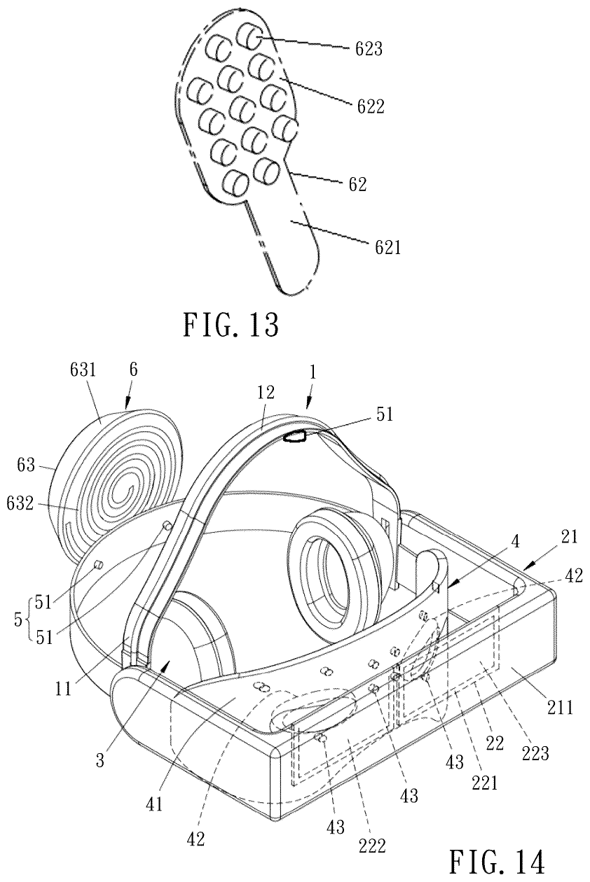

Referring to FIGS. 12-13, different from the first embodiment, the transcranial stimulation unit 6 provided by the second embodiment includes two first transcranial electrical stimulation media 62, and the two first transcranial electrical stimulation media 62 are respectively connected to the rear side part 13 to correspond to the P1 position and P2 position among the electrode positions of the International 10-20 EEG system. That is, compared with the first embodiment, the second embodiment is equivalent to replacing two first magnetic stimulation medium 61 with two first transcranial electrical stimulation media 62 in the first embodiment.

The first transcranial electrical stimulation media 62 comprises a second extension frame 621, a first connecting seat 622 and a plurality of first conductive posts 623, wherein the second extension frame 621 is connected to the rear side part 13, the first connecting seat 622 is connected to one end of the second extension frame 621 far away from the rear side part 13, and the first conductive posts 623 are connected to the first connecting seat 622 and electrically connected to the control unit 8.

In this embodiment, the first conductive posts 623 outputs a direct current, and the current can be the direct current or alternative current is between 0.01 mA-2 mA.

Preferably, the first transcranial electrical stimulation media 62 is detachably connected to the rear side part 13, and the first transcranial electrical stimulation media with different structures can be replaced as needed, and the first transcranial electrical stimulation media can also be replaced with a magnetic stimulation medium as needed.

It should be noted that the magnetic stimulation medium and the first transcranial electrical stimulation media are used alternatively.

Preferably, the first conductive post 623 is made of a silicon electrode material.

### Embodiment III

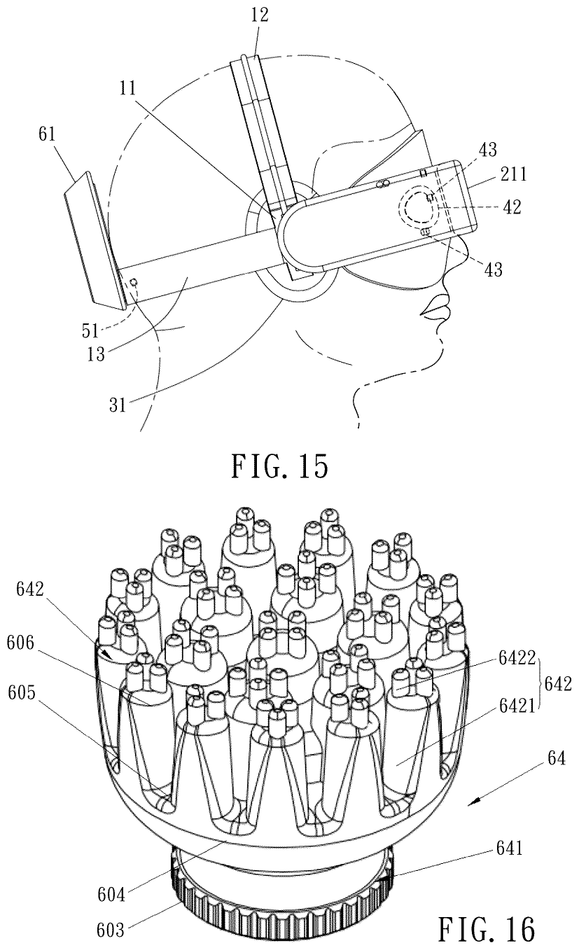

Referring to FIGS. 14-15, different from the first embodiment, the transcranial stimulation unit 6 provided by the third embodiment includes a second magnetic stimulation medium 63, and the second magnetic stimulation medium 63 includes a shell-shaped shell 631 and a second magnetic coil 632. The shell-shaped shell 631 is connected to the rear side part 13 to correspond to the occipital lobe of the user 9 and the brain region of the visual center, and the second magnetic coil 632 is connected to the shell-shaped shell 631 and arranged in a spiral shape. The second magnetic coil 632 is electrically connected to the control unit 8 and can generate a magnetic force when energized. That is, compared with the first embodiment, the third embodiment is equivalent to replacing two smaller first magnetic stimulation medium 61 in the first embodiment with a larger second magnetic stimulation medium 63. In the first embodiment, two first magnetic stimulation medium 61 can precisely magnetically stimulate the P1 position and P2 position of the user respectively, while in the third embodiment, a second magnetic stimulation medium 63 can magnetically stimulate a larger area of the occipital lobe of the user corresponding to the visual center.

### Embodiment IV

Referring to FIG. 16, different from the first embodiment, the transcranial stimulation unit 6 provided by the fourth embodiment includes two second transcranial electrical stimulation media 64, and the two second transcranial electrical stimulation media 64 are respectively connected to the rear side part to correspond to the P1 position and P2 position in the international 10-20 EEG electrode position.

The second transcranial electrical stimulation media is in the form of direct current output, and the current of direct current or alternative current is between 0.01 mA-2 mA.

Referring to FIG. 15, in this embodiment, the second transcranial electrical stimulation media comprises a second connecting seat 641 and a second conductive post 642, wherein the second connecting seat 641 has a third end 603 and a fourth end 604 opposite to each other, and the third end 603 is connected to the bracket unit 1, and the second conductive post 642 is connected to the fourth end 604 and electrically connected to the control unit 8.

The number of the second conductive posts 642 is multiple, and the second conductive posts 642 are distributed in parallel on the end face of the fourth end 604. A plurality of second conductive posts 642 are connected to the end face of the connecting seat 614, so that a single second conductive post 642 can have a smaller volume like comb, so that the single second conductive post 642 can avoid hair insulation to contact scalp of the user 9 and has smaller but many contact areas with the user 9, and the smaller but many contact areas can reduce the current density through the skin, which is helpful to reduce the potential stimulation risk or discomfort. At the same time, the plurality of second conductive posts 642 can form a plurality of contact positions with the user 9 and form a larger coverage area, thereby ensuring that the second transcranial electrical stimulation media can cover the P1 position or P2 position of the user 9.

In this embodiment, the end face of the fourth end 604 has a circular structure, and the center of the end face of the fourth end 604 is provided with a second conductive post 642. The end face of the fourth end 604 has a plurality of arrangement regions distributed at intervals along its radial direction, and each arrangement region has a plurality of second conductive posts 642 distributed around the center of the end face of the fourth end 604. In this way, a plurality of second conductive posts 642 can be densely arranged on the end face of the circular fourth end 604 and form a circular coverage area, so that the second transcranial electrical stimulation media 64 can completely cover the area to be stimulated by the user 9 and make part of the second conductive posts 642 precisely align with the P1 position or P2 position of the user 9, and further enable the current of the second conductive posts 642 to precisely stimulate the P1 position or P2 position of the user, so as to effectively increase visual acuity of the user.

Specifically, in this embodiment, the number of arrangement regions is two.

In other embodiments, the number of arrangement regions can also be any number such as one, three or four, which is not limited here.

The second conductive post 642 comprises a connecting part 6421 and a silicon electrode part 6422, wherein the connecting part 6421 has a fifth end 605 and a sixth end 606, the fifth end 605 is connected to the fourth end 604, and the silicon electrode part 6422 is connected to the sixth end 606. The number of silicon electrode part 6422 is plural, and the plurality of silicon electrode part 6422 are distributed in parallel on the end face of the sixth end 606. The adoption of a plurality of silicon electrode parts 6422 enables a single silicon electrode part 6422 to have a smaller volume, so that the single silicon electrode part 6422 has smaller but many contact areas with the user 9, and the smaller but many contact area can reduce the current density through the skin, which is helpful to reduce the potential stimulation risk or discomfort. At the same time, because the material of the silicone electrode part 6422 is soft, the silicone part 6422 can be in soft contact with the user 9, which is helpful to further reduce the potential stimulation risk or discomfort.

The silicon electrode part 6422 is made of silicon electrode material.

The material of the connecting part 6421 and the material of the silicon electrode part 6422 may be the same or different.

The above is only the preferred embodiment of the present invention, and it should be pointed out that a person skilled in the art can make several improvements and substitutions without departing from the technical principles of the present invention, and these improvements and substitutions should also be regarded as the protection scope of the present invention.

What is claimed is:

1. An amblyopia training device including:
a bracket unit corresponding to a user's head shape, and comprising a top side part, a rear side part and two ears part, wherein the two ears respectively correspond to the user's ears; two ends of the top side part are respectively connected with the two ears part and straddle the user's head upwards; and two ends of the rear side part are respectively connected with the two ears part and straddle the user's occipital bone backwards;
a virtual reality unit comprising a display headset connected to the bracket unit and capable of switching between a display mode and a visual photobiomodulatory stimulation mode, wherein when the display headset is in the display mode, the display headset displays a virtual image, and when the display headset is in the visual photobiomodulatory stimulation mode, the display headset displays flashing light with a specific wavelength and a specific frequency;
a dichoptic training unit with contrast color filters comprising a frame, a first contrast color filter and a second contrast color filter, wherein the frame is connected to the inner side of the display headset, and the first contrast color filter and the second contrast color filter are respectively connected to the frame to correspond to the two eyes of the user, and the colors of the first contrast color filter and the second contrast color filter are contrastly different;
a binaural audio stimulation unit comprising paired speakers, wherein the paired speakers are respectively connected to the two ears part and used for playing binaural beats music, and the binaural beats music has an binaural audio frequency difference;
a transcranial stimulation unit connected to the rear side part to correspond to the brain region of the occipital lobe of the user corresponding to the visual center;
a control unit electrically connected with the display headset, the speaker and the transcranial stimulation unit, wherein the control unit stores data information of the binaural beats music and the virtual image.

2. The amblyopia training device of claim 1, further comprising an eye mask unit and an head laser acupoint stimulation unit, wherein the eye mask unit comprises an eye mask body, two eyes surrounding heating parts and eyes surrounding acupoint stimulation parts, The eye mask body is detachably connected to the bracket unit and straddles the eyes of the user forward, the eye mask body is provided with eye holes for the user's eyes to be exposed and watch virtual reality, the dichoptic training unit with contrast color filters is located between the eye mask body and the display headset, and the eyes surrounding heating parts are electrically connected with the control unit. The eyes surrounding heating parts are connected to the eye mask body, the eyes surrounding acupoint stimulation parts are electrically connected to the control unit, and eyes surrounding acupoint stimulation parts are connected to the eye mask body and correspond to the user's two Jingming BL1 acupoints, two Cuanzhu BL2 acupoints, two Yuyao EX-HN4 acupoints, two Sizhukong TE23 acupoints and two Chengqi ST1 acupoints respectively; the head laser acupoint stimulation unit comprises three head laser acupoint stimulation medium, which are electrically connected with the control unit and used for outputting laser, and two head laser acupoint stimulation medium are connected to the rear and top side part to respectively correspond to two Fengchi GB20 acupoints and one Baihui GV20 acupoint of the user.

3. The amblyopia training device according to claim 2, wherein the binaural audio frequency difference of the binaural beats music is between 4 Hz and 18 Hz; the virtual image for dichoptic training unit comprises main two contrast color blocks matching contrast color filters in claim 2, a first color block and a second contrast color block, wherein the colors of the main color block are contrastly different from or as the same as those of the first color block as well as the colors of the main color block are contrastly as the same as or different from those of the second color block, respectively; the flashing light of visual photobiomodulatory stimulation with a specific wavelength and a specific frequency is light with a wavelength between 380 nm and 780 nm and a frequency between 5 Hz and 45 Hz; the laser wavelength of the head laser acupoint stimulation medium is 650-808 nm.

4. The amblyopia training device according to claim 1, further comprising an exercise bike unit which comprises a bike frame, a flywheel and a pedal group, wherein the bike frame is used for the user to ride, the flywheel is rotatably connected to the bike frame, and the pedal group is in transmission connection with the flywheel and is used for the user to step on it to make the flywheel rotate around the central axis of the flywheel.

5. The amblyopia training device according to claim 1, wherein the transcranial stimulation unit comprises two first magnetic stimulation medium, and the two first magnetic stimulation medium are respectively connected to the rear side part to correspond to the P1 position and the P2 position among the positions of the international 10-20 EEG electrodes; the first magnetic stimulation medium comprises a first extension frame, a mounting seat and a first magnetic coil, wherein the first extension frame is connected to the rear side part ; the mounting seat has a first end and a second end which are opposite; the first end is connected to the first extension frame; and the first magnetic coil is connected to the mounting seat and electrically connected with the control unit.

6. The amblyopia training device according to claim 5, wherein the mounting seat comprises a plurality of support layers which are sequentially stacked along the direction from the first end to the second end, and each support layer is provided with the first magnetic coil; among any two adjacent support layers, the outer contour of one support layer close to the first end is larger than that of one support layer far from the first end, and the one support layer far from the first end is arranged in the middle of one support layer close to the first end.

7. The amblyopia training device according to claim 6, wherein an end face of the support layer far from the first end is a first magnetic surface, and the first magnetic coil surrounds a first area around the center of the first magnetic surface on the first magnetic surface; among any two adjacent support layers, one support layer far from the first end is arranged in the first area of one support layer close to the first end.

8. The amblyopia training device according to claim 1, wherein the transcranial stimulation unit comprises two first transcranial electrical stimulation media, and the two first transcranial electrical stimulation media are respectively connected to the rear side part to respectively correspond to the P1 position and P2 position in the international 10-20 EEG electrode position; the first transcranial electrical stimulation media comprises a second extension frame, a first connecting seat and a plurality of first conductive posts, the second extension frame is connected to the rear side part, the first connecting seat is connected to one end of the second extension frame far away from the rear side part, and the first conductive posts are connected to the first connecting seat and electrically connected with the control unit.

9. The amblyopia training device according to claim 1, wherein the transcranial stimulation unit comprises two second magnetic stimulation mediums, and the second magnetic stimulation mediums comprise a shell and a second magnetic coil, and the shell is connected to the rear side part, and the second magnetic coil is connected to the shell and arranged in a spiral shape.

10. The amblyopia training device according to claim 1, wherein the transcranial stimulation unit comprises two second transcranial electrical stimulation media, and the two second transcranial electrical stimulation media are respectively connected to the rear side part to correspond to the P1 position and P2 position among the positions of the international 10-20 EEG electrodes; The second transcranial electrical stimulation media comprises a second connecting seat and a second conductive post, wherein the second connecting seat is provided with a third end and a fourth end which are opposite, the third end is connected to the bracket unit, and the second conductive post is connected to the fourth end and electrically connected with the control unit; the number of the second conductive posts is multiple, and the second conductive posts are distributed on the end face of the fourth end in parallel; the second conductive column comprises a connecting part and a silicon electrode part, wherein the connecting part is provided with a fifth end and a sixth end which are opposite, the fifth end is connected to the fourth end, and the silicon electrode part is connected to the sixth end; the number of the silicon electrode part is multiple, and the silicon electrode part are distributed on the end face of the sixth end in parallel.
